(19) United States
(12) Patent Application Publication
(10) Pub. No.: US 2011/0144064 A1
(43) Pub. Date: Jun. 16, 2011

(54) Inhibitors of Kynurenine Aminotransferase and Uses Therefor

(76) Inventors: Robert Schwarcz, Baltimore, MD (US); Roberto Pellicciari, Perugia (IT); Timothy Gately, San Francisco, CA (US)

(21) Appl. No.: 12/085,931

(22) PCT Filed: Nov. 30, 2006
(86) PCT No.: PCT/US2006/045828

§ 371 Date: Feb. 23, 2010

Related U.S. Application Data
(60) Provisional application No. 60/740,972, filed on Nov. 30, 2005.

(51) Int. Cl. A61K 31/662; C12N 5/071; A61K 31/197; A61K 31/19; G01N 33/50; C07C 53/08; C07C 55/02; C07C 309/63; C07F 9/38; A61P 25/00

(52) U.S. Cl. 514/143; 435/325; 514/561; 514/574; 514/557; 436/129; 436/104; 436/111; 436/120; 562/512; 562/590; 562/30; 562/24

(57) ABSTRACT

Provided herein are methods of decreasing a level of kynurenic acid in a cell and of treating a pathophysiological condition in a subject associated with an increase in kynurenic acid in a subject. In these methods the inhibitory action of dicarboxylic acids or derivatives or analogs thereof are effective to inhibit activity of kynurenine aminotransferase II. Also provided is a method of screening for potential inhibitory compounds for kynurenine aminotransferase II. The dicarboxylic acids or derivatives or analogs thereof may have the structural formula, where R¹ is H, NH₂ or NHCH₃, R² is H or CH₃, n is 0 to 14, and X is —COOH, CH₂OH, —PO₃H₂, —SO₃H, or —SO₃H; or a pharmacologically acceptable salt.

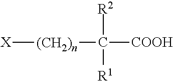

### BACKGROUND OF THE INVENTION

1. Field of the Invention

The present invention relates generally to the fields of organic chemistry, enzymology and diseases and disorders involving the brain. More specifically, the present invention relates to inhibitors of kynurenine aminotransferases and their uses as agents against cognitive, neurodegenerative and psychiatric disorders and diseases.

2. Description of the Related Art

Kynurenic acid is an endogenous product of tryptophan degradation along the kynurenine pathway. It is a neuroactive compound with antagonist activity at the glycine co-agonist site of the N-methyl-D-aspartate (NMDA) receptor and the alpha-7-nicotinic receptor. It can act as an endogenous modulator of excitatory neurotransmission and has been implicated in the pathogenesis of several neurological and psychiatric diseases.

A decrease in the levels of kynurenic acid in the brain will increase the activity of these receptors. An increase in the activity of these receptors has been proven in animal models to ameliorate several brain diseases, such as schizophrenia, ADHD, bipolar disease, depression, obsessive compulsive disorder, drug addiction, mental retardation and other neurodevelopmental disorders. Moreover, these receptors have been implicated in memory and cognitive processes and in plasticity, neuronal regeneration and aging. U.S. Pat. No. 5,786,508 discloses derivatives of kynurenine that are promulgated in the treatment of cognitive disorders associated with the aging processes of the brain and perinatal brain disorders.

Certainly there is a recognized need for improvement in the art in methods of treating or alleviating symptoms associated with cognitive and psychiatric disorders and diseases where pathogenesis is associated with an increase in kynurenic acid levels. Thus, the prior art is still deficient in the lack of methods of inhibiting kynurenic acid synthesis. Specifically, the prior art is deficient in the lack of methods of inhibiting kynurenine aminotransferase II activity to reduce levels of kynurenic acid using dicarboxylic acid compounds or derivatives or analogs thereof. The present invention fulfills this long-standing need and desire in the art.

### SUMMARY OF THE INVENTION

The present invention is directed to a method of decreasing a level of kynurenic acid in a cell. The method comprises contacting a cell exhibiting kynurenine aminotransferase II activity with an amount of a dicarboxylic acid compound or a derivative or analog thereof effective to inhibit synthesis of kynurenic acid by the kynurenine aminotransferase II activity thereby decreasing the level of kynurenic acid in the cell. In this method, the dicarboxylic acid, derivative or analog thereof may have the structural formula:

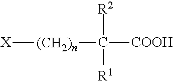

where R¹ is H, NH₂ or NHCH₃, R² is H or CH₃, n is 0 to 14, and X is —COOH, —CH₂OH, —PO₃H₂, —SO₂H, or —SO₃H or a pharmacologically acceptable salt. The dicarboxylic acid or derivative or analog thereof may be a racemate (DL) or a levarotatory (L) isomer thereof.

The present invention also is directed to a method of treating a pathophysiological condition associated with an increase in kynurenic acid in a subject. The method comprises administering to the subject a pharmacologically effective amount of a dicarboxylic acid or a derivative or analog thereof as described herein.

The present invention is directed further to a method for identifying an inhibitory compound of kynurenine aminotransferase II activity. The method comprises selecting a test compound and measuring the level of kynurenic acid in the presence or absence of the test compound. The level of kynurenic acid in the presence of the test compound is compared with the level of kynurenic acid in the absence of the test compound. A decrease in kynurenic acid in the presence of the test compound is indicative that the test compound has an ability to inhibit kynurenine aminotransferase II activity.

The present invention is directed further yet to an inhibitory compound or a pharmaceutical composition comprising the same screened by the method described herein.

Other and further aspects, features, benefits, and advantages of the present invention will be apparent from the following description of the presently preferred embodiments of the invention given for the purpose of disclosure.

### DETAILED DESCRIPTION OF THE INVENTION

### I. Definitions

The use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

As used herein, the term “compound” is interchangeable with “inhibitor”, or “inhibitory compound” and means a molecular entity of natural, semi-synthetic or synthetic origin that blocks, stops, inhibits, and/or suppresses substrate interactions with a kynurenine aminotransferase II, particularly the formation of kynurenic acid from kynurenine.

As used herein, the term “inhibit” refers to the ability of the compound to block, partially block, interfere, decrease, reduce or deactivate a kynurenine aminotransferase II. Thus, one of skill in the art understands that the term inhibit encompasses a complete and/or partial loss of activity of a kynurenine aminotransferase II. Such activity may be inhibited by preventing transamination of the substrate, by disruption of the interaction with the substrate, by sequestering kynurenine aminotransferase II and/or the substrate, or by other means. For example, a complete and/or partial loss of activity of the kynurenine aminotransferase II as may be indicated by a reduction in transamination of the substrate, a reduction in production of kynurenic acid, a reduction in cell proliferation, or the like.

As used herein, the term “contacting” refers to any suitable method of bringing one or more of the compounds described herein or other inhibitory agent into contact with a kynurenine aminotransferase II protein or polypeptide or fragment thereof or a cell comprising the same. In vitro or ex vivo this is achieved by exposing the kynurenine aminotransferase II protein or polypeptide or fragment thereof or cells comprising the same to the inhibitory agent in a suitable medium. For in vivo applications, any known method of administration is suitable as described herein.

As used herein, the terms “effective amount” or “therapeutically effective amount” are interchangeable and refer to an amount that results in an improvement or remediation of the symptoms of the disease or condition. Those of skill in the art understand that the effective amount may improve the patient's or subject's condition, but may not be a complete cure of the disease and/or condition.

As used herein, the terms “treating” or “treatment” includes prophylactic treatment as well as alleviation of ongoing or intermittent pathophysiological or pathoneurological symptoms occurring in a neurodegenerative, neurocognitive or psychiatric disease or disorder.

As used herein, the term “decreasing a level of kynurenic acid” refers to a reduction of available kynurenic acid in a cell due to the inhibitory action of a kynurenine aminotransferase II inhibitor on kynurenine aminotransferase II enzyme activity necessary for the formation of kynurenic acid from kynurenine.

As used herein, the term “dicarboxylic acid” shall refer to a C₂-C₁₇ dicarboxylic acid. A dicarboxylic acid derivative comprises one or more moieties substituted on the alkyl chain, preferably a methyl and/or amine moiety, more preferably substituted at C2. A dicarboxylic acid analog comprises the dicarboxylic acid or dicarboxylic acid derivative further substituting terminal carboxylic acid functional group of the C₂-C₁₇ dicarboxylic acid with an analogous group, preferably a methanol, a sulfinic acid moiety, a sulfonic acid moiety, or a phosphoric acid moiety.

As used herein, the term “subject” refers to any target of the treatment.

### II. Present Invention

In one embodiment of the present invention there is provided a method of decreasing a level of kynurenic acid in a cell, comprising contacting a cell exhibiting kynurenine aminotransferase II activity with an amount of a dicarboxylic acid compound or a derivative or analog thereof effective to inhibit synthesis of kynurenic acid by the kynurenine aminotransferase II activity thereby decreasing the level of kynurenic acid in the cell where the dicarboxylic acid compound or the derivative or analog thereof may have the structural formula:

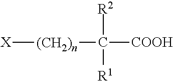

where R¹ is H, NH₂ or NHCH₃; R² is H or CH₃; n is 0 to 14; and X is —COOH, —CH₂OH, —PO₃H₂, —SO₂H, or —SO₃H; or a pharmacologically acceptable salt thereof. Alternatively, the administered compound may comprise a pharmaceutical composition including a pharmaceutically acceptable carrier.

In this embodiment the dicarboxylic acid compound or derivative or analog thereof may be 1,6-hexanedioic acid, 1,7-heptanedioic acid, 1,12-dodecanedioic acid, 1,13-tridecanedioic acid, 1,14-tetradecanedioic acid, 1,15-pentadecanedioic acid, 1,16-hexadecanedioic acid, 1,9-nonanedioic acid, 1,10-decanedioic acid, 1,11-undecanedioic acid, 16-hydroxyhexadecanoic acid, 2-amino-7-phosphonoheptanoic acid, cysteinesulfinic acid, homocysteinesulfinic acid, 2-amino-3-sulfopropionic acid, or 2-amino-4-sulfobutyric acid.

Also in this embodiment the dicarboxylic acid derivative or analog may be a racemate or a levarotatory isomer. Representative examples of racemates are DL-2-amino-1,4-butanedioic acid, DL-2-amino-1,5-pentanedioic acid, DL-2-amino-1,6-hexanedioic acid, DL-2-amino-1,7-heptanedioic acid, DL-2-amino-1,8-octanedioic acid, DL-2-amino-1,9-nonanedioic acid, DL-2-amino-1,11-undecanedioic acid, DL-2-amino-1,12-dodecandioic acid, DL-2-amino-1,13-tridecanedioic acid, DL-2-amino-1,16-hexadecanedioic acid, DL-2-amino-2-methyl-1,16-hexadecanedioic acid, DL-2-methylamino-1,16-hexadecanedioic acid, DL-2-amino-1,17-heptadecanedioic acid, DL-2-amino-4-phosphonobutyric acid, DL-2-amino-5-phosphonopentanoic acid, or DL-2-amino-6-phosphonohexaanoic acid. A representative example of a levarotatory isomer is L-2-amino-1,16-hexadecanedioic acid. Alternatively, L-2-amino-1,16-hexadecanedioic acid may comprise a pharmaceutical composition including a pharmaceutically acceptable carrier.

In one aspect of this embodiment R² is H and X is COOH in said dicarboxylic acid compound or derivative thereof. Examples of these compounds are 1,6-hexanedioic acid, 1,7-heptanedioic acid, 1,12-dodecanedioic acid, 1,13-tridecanedioic acid, 1,14-tetradecanedioic acid, 1,15-pentadecanedioic acid, 1,16-hexadecanedioic acid, DL-2-amino-1,4-butanedioic acid, DL-2-amino-1,5-pentanedioic acid, DL-2-amino-1,6-hexanedioic acid, DL-2-amino-1,7-heptanedioic acid, DL-2-amino-1,8-octanedioic acid, DL-2-amino-1,9-nonanedioic acid, DL-2-amino-1,11-undecanedioic acid, 1,9-nonanedioic acid, 1,10-decanedioic acid, 1,11-undecanedioic acid, DL-2-amino-1,12-dodecandioic acid, DL-2-amino-1,13-tridecanedioic acid, DL-2-amino-1,16-hexadecanedioic acid, L-2-amino-1,16-hexadecanedioic acid, DL-2-amino-1,17-heptadecanedioic acid, or DL-2-methylamino-1,16-hexadecanedioic acid.

In another aspect R² is CH₃ and X is COOH in the dicarboxylic acid derivative. An example of this dicarboxylic acid derivative is DL-2-amino-2-methyl-1,16-hexadecanedioic acid. In yet another aspect of this embodiment R¹ is H or NH₂ and R² is H in the dicarboxylic acid analog. Examples of dicarboxylic acid analogs are 16-hydroxyhexadecanoic acid, DL-2-amino-4-phosphonobutyric acid, DL-2-amino-5-phosphonopentanoic acid, DL-2-amino-6-phosphonohexaanoic acid, 2-amino-7-phosphonoheptanoic acid, cysteinesulfinic acid, homocysteinesulfinic acid, 2-amino-3-sulfopropionic acid, or 2-amino-4-sulfobutyric acid.

Also in this embodiment the level of kynurenic acid may be associated with a cognitive disease or disorder in a subject. Examples of a cognitive disease or disorder are is mental retardation, is associated with Alzheimer's Disease, is associated with Parkinson's Disease, a neurodegenerative disease or seizure disorders, or an age-related cognitive deficit. Alternatively, the level of kynurenic acid may be associated with a psychiatric disease or disorder in a subject. Examples of psychiatric diseases or disorders are schizophrenia, attention-deficit hyperactivity disorder, bipolar disease, depression, an obsessive-compulsive disorder, or drug addiction.

In another embodiment of the present invention there is provided a method of treating a pathophysiological condition associated with an increase in kynurenic acid in a subject, comprising administering to the subject a pharmacologically effective amount of the dicarboxylic acids or derivatives or analogs thereof having the structural formulas as described supra. Furthermore, the specific dicarboxylic acids or derivatives or analogs thereof may be those specific compounds as described supra.

In this embodiment the pathophysiological condition may be a cognitive disease or disorder. Alternatively, the pathophysiological condition may be a psychiatric disease or disorder. Examples of a cognitive disease or disorder or of psychiatric diseases or disorders are as described supra.

In yet another embodiment of the present invention there is provided a method of screening for a potential inhibitory compound of kynurenine aminotransferase II activity, comprising selecting a test compound; measuring the level of kynurenic acid in the presence or absence of the test compound; and comparing the level of kynurenic acid in the presence of the test compound with the level of kynurenic acid in the absence of the test compound, where a decrease in kynurenic acid in the presence of the test compound is indicative that the compound has an ability to inhibit kynurenine aminotransferase II activity.

In an aspect of this embodiment the inhibitory compound may have the structural formula as described supra. More specifically, the inhibitory compound may be a derivative or analog of the dicarboxylic acids or derivatives or analogs thereof as described supra.

In this embodiment the screened inhibitory compound is effective to reduce levels of kynurenic acid associated with a cognitive disease or disorder due to aging or associated with a psychiatric disease or disorder. Examples of a cognitive disease or disorder or of psychiatric diseases or disorders are as described supra.

In yet another embodiment of the present invention there is provided an inhibitory compound screened by the method described supra. Further to this embodiment the inhibitory compound and a pharmaceutically acceptable carrier may comprise a pharmaceutical composition.

The present invention provides methods of inhibiting an activity of the kynurenine aminotransferase II enzyme in vitro and in vivo using an inhibitory compound to inhibit the transamination of kynurenine to kynurenic acid. These kynurenine aminotransferase II enzyme inhibitors are dicarboxylic acids or derivatives or analogs thereof. Generally, the dicarboxylic acids may comprise an alkyl chain 1 to 15 carbons in length. The alkylene chain may comprise one or two short alkyl, e.g., methyl, amine or methylamine substituents and/or the C₂-C₁₇ terminal carboxylic acid functional group may be substituted with an analogous non-carbon acid or alcohol moiety.

The dicarboxylic acids and derivatives or analogs thereof useful in the methods described herein may have a general structure of:

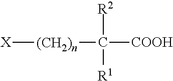

R¹ may be hydrogen, amine or methylamine, R² may be hydrogen or methyl and X may be a carboxylic acid moiety, methanol, a sulfinic acid moiety, a sulfonic acid moiety, or a phosphoric, also known as phosphonic, acid moiety. Furthermore, the racemate or separate optical isomers thereof, preferably the levarotatory isomer, may be used as therapeutics in the methods presented herein. Some of the dicarboxylic acid compounds, derivatives and analogs disclosed herein are commercially available as indicated or otherwise may be synthesized by methods well known in the art. It is particularly contemplated that compounds where X is any disclosed substituent, R¹ is hydrogen, amine or methylamine, R² is hydrogen or methyl, such that R¹ and R² are not both hydrogen and n is 4 to 14 are novel compounds.

These compounds also may comprise a salt with a pharmacologically acceptable base such as, but not limited to, an alkali metal, e.g. sodium or potassium, an alkaline-earth metal, e.g. calcium or magnesium, zinc or aluminium, or organic bases, such as aliphatic amines, e.g., methylamine, diethylamine, trimethylamine, ethylamine and heterocyclic amines such as piperidine. These compounds or salts thereof may be formulated as pharmaceutical compositions comprising a pharmaceutically acceptable carrier as is known and standard in the art.

Representative dicarboxylic acids may be, but not limited to, 1,6-hexanedioic acid, 1,7-heptanedioic acid, 1,12-dodecanedioic acid, 1,13-tridecanedioic acid, 1,14-tetradecanedioic acid, 1,15-pentadecanedioic acid, 1,16-hexadecanedioic acid, 1,9-nonanedioic acid, 1,10-decanedioic acid, or 1,11-undecanedioic acid.

Representative racemate (DL) derivatives of the dicarboxylic acids may be, but not limited to, DL-2-amino-1,4-butanedioic acid, DL-2-amino-1,5-pentanedioic acid, DL-2-amino-1,6-hexanedioic acid, DL-2-amino-1,7-heptanedioic acid, DL-2-amino-1,8-octanedioic acid, DL-2-amino-1,9-nonanedioic acid, DL-2-amino-1,11-undecanedioic acid, DL-2-amino-1,12-dodecandioic acid, DL-2-amino-1,13-tridecanedioic acid, DL-2-amino-1,16-hexadecanedioic acid, DL-2-amino-2-methyl-1,16-hexadecanedioic acid, DL-2-methylamino-1,16-hexadecanedioic acid, or DL-2-amino-1,17-heptadecanedioic acid. A representative levarotatory (L) derivative of the dicarboxylic acids may be, but not limited to, L-2-amino-1,16-hexadecanedioic acid.

Representative analogs of the dicarboxylic acids may be, but not limited to, 16-hydroxyhexadecanoic acid, 2-amino-7-phosphonoheptanoic acid, cysteinesulfinic acid, homocysteinesulfinic acid, 2-amino-3-sulfopropionic acid, or 2-amino-4-sulfobutyric acid. Representative racemate (DL) analogs of the dicarboxylic acids may be, but not limited to, DL-2-amino-4-phosphonobutyric acid, DL-2-amino-5-phosphonopentanoic acid, or DL-2-amino-6-phosphonohexaanoic acid.

It is contemplated that inhibitory compounds of kynurenine aminotransferase II may be identified using suitable assays known and standard in the art. A suitable inhibitor would inhibit the transamination of kynurenine to kynurenic acid by kynurenine aminotransferase II. A simple decrease in kynurenic acid levels in the presence of the potential inhibitory or test compound is indicative the potential inhibitor is effective to inhibit enzyme activity. For example, inhibitory compounds may be designed based on the structures of the dicarboxylic acids or derivatives or analogs thereof. Alternatively, inhibitory compounds may be designed based on the structure of the human kynurenine aminotransferase II enzyme or homologs thereof using in part computer aided design as is known in the art.

As inhibitors of the kynurenine aminotransferase II enzyme, these dicarboxylic acid compounds, derivatives and analogs are useful to prevent or to treat diseases and disorders of the brain in which an increase in kynurenic acid is associated therewith. It is contemplated that these compounds will have a prophylactic and/or therapeutic effect on diseases and disorders affecting cognition and/or memory or psychiatric diseases and disorders. For example cognitive diseases and disorders may be cognitive disorders in children, mental retardation, disorders associated with Alzheimer's Disease, disorders associated with Parkinson's Disease, disorders in neurodegenerative disease and seizure disorders, and age-related cognitive deficit. Psychiatric diseases and disorders include schizophrenia, attention-deficit hyperactivity disorder (ADHD), bipolar disease, depression, obsessive compulsive disorders or drug addiction.

An effective amount of an kynurenine aminotransferase II inhibitor that may be administered to a cell includes a dose of about 0.0001 nM to about 2000 mM. More specifically, doses of an agonist to be administered are from about 0.01 nM to about 2000 mM; about 0.01 mM to about 0.05 mM; about 0.05 mM to about 1.0 mM; about 1.0 mM to about 1.5 mM; about 1.5 mM to about 2.0 mM; about 2.0 mM to about 3.0 mM; about 3.0 mM to about 4.0 mM; about 4.0 mM to about 5.0 mM; about 5.0 mM to about 10 mM; about 10 mM to about 50 mM; about 50 mM to about 100 mM; about 100 mM to about 200 mM; about 200 mM to about 300 mM; about 300 mM to about 500 mM; about 500 mM to about 1000 mM; about 1000 mM to about 1500 mM and about 1500 mM to about 2000 mM. Of course, all of these amounts are exemplary, and any amount in-between these points is also expected to be of use in the invention.

The kynurenine aminotransferase II inhibitor or related-compound (derivative) thereof can be administered parenterally or alimentary. Parenteral administrations include, but are not limited to intravenously, intradermally, intramuscularly, intraarterially, intrathecally, subcutaneous, or intraperitoneally U.S. Pat. Nos. 6,613,308, 5,466,468, 5,543,158; 5,641,515; and 5,399,363 (each specifically incorporated herein by reference in its entirety). Alimentary administrations include, but are not limited to orally, buccally, rectally, or sublingually.

The administration of the therapeutic compounds and/or the therapies of the present invention may include systemic, local and/or regional administrations, for example, topically (dermally, transdermally), via catheters, implantable pumps, etc. Alternatively, other routes of administration are also contemplated such as, for example, arterial perfusion, intracavitary, intraperitoneal, intrapleural, intraventricular and/or intrathecal. The skilled artisan is aware of determining the appropriate administration route using standard methods and procedures. Other routes of administration are discussed elsewhere in the specification and are incorporated herein by reference.

Treatment methods will involve treating an individual with an effective amount of a composition containing kynurenine aminotransferase II inhibitor or related-compound thereof. An effective amount is described, generally, as that amount sufficient to detectably and repeatedly to ameliorate, reduce, minimize or limit the extent of a disease or its symptoms. More specifically, it is envisioned that the treatment with the kynurenine aminotransferase II inhibitor or related-compounds thereof will inhibit kynurenine aminotransferase II transamination of a kynurenine substrate, wherein the kynurenine substrate would have been transaminated by the kynurenine aminotransferase II to produce kynurenic acid if not for the inhibition.

The effective amount of kynurenine aminotransferase II inhibitor or related-compounds thereof to be used are those amounts effective to produce beneficial results in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As is well known in the art, a specific dose level of active compounds such as kynurenine aminotransferase II inhibitor or related-compounds thereof for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

One of skill in the art realizes that the effective amount of the kynurenine aminotransferase II inhibitor or related-compound thereof can be the amount that is required to achieve the desired result, that is, although not limited to, reduction/inhibition in transamination of kynurenine.

Administration of the therapeutic kynurenine aminotransferase II inhibitor composition of the present invention to a patient or subject will follow general protocols for the administration of therapies used in treatment of diseases or disorders of the brain taking into account the toxicity, if any, of the kynurenine aminotransferase II inhibitor. It is expected that the treatment cycles would be repeated as necessary. It also is contemplated that various standard therapies may be applied in combination with the described therapy.

### Pharmaceutical Formulations and Methods of Treating

### Compositions of the Present Invention

The present invention also contemplates therapeutic methods employing compositions comprising the active substances disclosed herein. Preferably, these compositions include pharmaceutical compositions comprising a therapeutically effective amount of one or more of the active compounds or substances along with a pharmaceutically acceptable carrier.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

### Dose Determinations

By a “therapeutically effective amount” or simply “effective amount” of an active compound, such as the dicarboxylic acids, analogs and derivatives provided herein, is meant a sufficient amount of the compound to treat a disease or disorder of the brain, as described, at a reasonable benefit/risk ratio applicable to any medical treatment. It will be understood, however, that the total daily usage of the active compounds and compositions of the present invention will be decided by the attending physician within the scope of sound medical judgment. The specific therapeutically effective dose level for any particular patient will depend upon a variety of factors including the disorder, disease or injury being treated and the severity of the disorder, disease or injury; activity of the specific compound employed; the specific composition employed; the age, body weight, general health, sex and diet of the patient; the time of administration, route of administration, and rate of excretion of the specific compound employed; the duration of the treatment; drugs used in combination or coinciding with the specific compound employed; and like factors well known in the medical arts.

Toxicity and therapeutic efficacy of such compounds can be determined by standard pharmaceutical procedures in cell assays or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell based assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The total daily dose of the active compounds of the present invention administered to a subject in single or in divided. doses can be in amounts, for example, from 0.01 to 25 mg/kg body weight or more usually from 0.1 to 15 mg/kg body weight. Single dose compositions may contain such amounts or submultiples thereof to make up the daily dose. In general, treatment regimens according to the present invention comprise administration to a human or other mammal in need of such treatment from about 1 mg to about 1000 mg of the active substance(s) of this invention per day in multiple doses or in a single dose of from 1 mg, 5 mg, 10 mg, 100 mg, 500 mg or 1000 mg.

### Formulations and Administration

The compounds of the present invention may be administered alone or in combination or in concurrent therapy with other agents which affect the targeted cell(s). Liquid dosage forms for oral administration may include pharmaceutically acceptable emulsions, microemulsions, solutions, suspensions, syrups and elixirs containing inert diluents commonly used in the art, such as water, isotonic solutions, or saline. Such compositions may also comprise adjuvants, such as wetting agents; emulsifying and suspending agents; sweetening, flavoring and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulation can be sterilized, for example, by filtration through a bacteria-retaining filter, or by incorporating sterilizing agents in the form of sterile solid compositions, which can be dissolved or dispersed in sterile water or other sterile injectable medium just prior to use.

In order to prolong the effect of a drug, it is often desirable to slow the absorption of a drug from subcutaneous or intramuscular injection. The most common way to accomplish this is to inject a suspension of crystalline or amorphous material with poor water solubility. The rate of absorption of the drug becomes dependent on the rate of dissolution of the drug, which is, in turn, dependent on the physical state of the drug, for example, the crystal size and the crystalline form. Another approach to delaying absorption of a drug is to administer the drug as a solution or suspension in oil. Injectable depot forms can also be made by forming microcapsule matrices of drugs and biodegradable polymers, such as polylactide-polyglycoside. Depending on the ratio of drug to polymer and the composition of the polymer, the rate of drug release can be controlled. Examples of other biodegradable polymers include polyorthoesters and polyanhydrides. The depot injectables can also be made by entrapping the drug in liposomes or microemulsions, which are compatible with body tissues.

Suppositories for rectal administration of the drug can be prepared by mixing the drug with a suitable non-irritating excipient, such as cocoa butter and polyethylene glycol which are solid at ordinary temperature but liquid at the rectal temperature and will, therefore, melt in the rectum and release the drug.

Solid dosage forms for oral administration may include capsules, tablets, pills, powders, gelcaps and granules. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such as magnesium stearate and microcrystalline cellulose. In the case of capsules, tablets and pills, the dosage forms may also comprise buffering agents. Tablets and pills can additionally be prepared with enteric coatings and other release-controlling coatings. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferably, in a certain part of the intestinal tract, optionally in a delayed manner. Examples of embedding compositions which can be used include polymeric substances and waxes.

Dosage forms for topical or transdermal administration of a compound of this invention further include ointments, pastes, creams, lotions, gels, powders, solutions, sprays, inhalants or patches. Transdermal patches have the added advantage of providing controlled delivery of active compound to the body. Such dosage forms can be made by dissolving or dispersing the compound in the proper medium. Absorption enhancers can also be used to increase the flux of the compound across the skin. The rate can be controlled by either providing a rate controlling membrane or by dispersing the compound in a polymer matrix or gel. The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

The method of the present invention employs the compounds identified herein for both in vitro and in vivo applications. For in vivo applications, the invention compounds can be incorporated into a pharmaceutically acceptable formulation for administration. Those of skill in the art can readily determine suitable dosage levels when the invention compounds are so used. As employed herein, the phrase “suitable dosage levels” refers to levels of compound sufficient to provide circulating concentrations high enough to effectively inhibit kynurenine aminotransferase II activity and to reduce transamination of kynurenine.

In accordance with a particular embodiment of the present invention, compositions comprising at least one kynurenine aminotransferase II inhibitory compound (for example, the dicarboxylic acids, as described above), and a pharmaceutically acceptable carrier are contemplated. Exemplary pharmaceutically acceptable carriers include carriers suitable for oral, intravenous, subcutaneous, intramuscular, intracutaneous, and the like administration. Administration in the form of creams, lotions, tablets, dispersible powders, granules, syrups, elixirs, sterile aqueous or non-aqueous solutions, suspensions or emulsions, and the like, is contemplated.

For the preparation of oral liquids, suitable carriers include emulsions, solutions, suspensions, syrups, and the like, optionally containing additives such as wetting agents, emulsifying and suspending agents, sweetening, flavoring and perfuming agents, and the like.

For the preparation of fluids for parenteral administration, suitable carriers include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized, for example, by filtration through a bacteria-retaining filter, by incorporating sterilizing agents into the compositions, by irradiating the compositions, or by heating the compositions. They can also be manufactured in the form of sterile water, or some other sterile injectable medium immediately before use. The active compound is admixed under sterile conditions with a pharmaceutically acceptable carrier and any needed preservatives or buffers as may be required.

The treatments may include various “unit doses.” Unit dose is defined as containing a predetermined quantity of the therapeutic composition calculated to produce the desired responses in association with its administration, e.g., the appropriate route and treatment regimen. The quantity to be administered, and the particular route and formulation, are within the skill of those in the clinical arts. Also of import is the subject to be treated, in particular, the state of the subject and the protection desired.

The following examples are given for the purpose of illustrating various embodiments of the invention and are not meant to limit the present invention in any fashion.

### Example 1

### Synthesis of DL-2-amino-1,16-hexadecanedioic acid

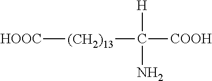

Starting product 15-hydroxypentadecanoic acid is esterified with methanol in the presence of boron trifluoride (BF₃) for 15 min. The obtained 15-hydroxypentadecanoic acid methyl ester is oxidized in position 15 to the aldehyde using pyridinium chlorochromate in dichloromethane for 3 hours. The 14-formyltetradecanoic acid methyl ester is then reacted with potassium cyanide and ammonium chloride in 28% ammonium hydroxide and methanol overnight to obtain the DL-15 carbomethoxy-2-aminopentadecane-1-nitrile. The DL-15-carbomethoxy-2-aminopentadecane-1-nitrile is then hydrolyzed overnight in HCl to obtain the DL-2-amino-1-16-hexadecanedioic acid.

This synthetic scheme may be used to synthesize other C2-amine substituted dicarboxylic acid derivatives by varying the length of the alkyl chain of the starting product.

### Example 2

### Inhibition of Kynurenine Aminotransferase II Activity by Dicarboxylic Acid Derivatives and Analogs

Compounds were tested as inhibitors of kynurenine aminotransferase II and the IC₅₀ values determined. For the determination of the KAT II activity, 80 μl of rat liver partially purified KAT II was incubated (2 hrs at 37° C. with 100 μl of 150 mM Tris-acetate (pH 7.4), 2 μM kynurenine, (2.5 nCi) [³H]-kynurenine 1 mM pyruvate and 70 μM pyridoxal-5′-phosphate. Add 20 μl of the inhibitor solution (10×). The reaction was terminated by the addition of 14 μl of 50% trichloroacetic acid and 1 ml of 0.1 N hydrochloric acid. The denaturated protein was removed by centrifugation and 1 ml of the supernatant was applied to a Dowex 50 W H⁺ cation exchange column which was then washed with 1 ml of 0.1 N hydrochloric acid followed by 1 ml of ultrapure water. [³H]-Kynurenic acid formed was subsequently eluted with 2×1 ml of ultrapure water and radioactivity was quantified by liquid scintillation spectrometry.

All compounds tested are commercially available except for compounds in bold that were synthesized using methods analogous to the synthetic scheme in Example 1. All compounds are in the racemic form, when applicable, unless indicated otherwise. Other untested compounds are commercially available or easily synthesized using standard synthetic methods. Data are presented as IC₅₀ values in μM for all tested compounds. Blanks are untested compounds.

Table 1 test compounds: R² is H, X is COOH and R¹ varies.

TABLE 1

| n | R¹ = H | R¹ = NH² |
|---|---|---|
| 0 | inactive | |
| 1 | inactive | 2456 |
| 2 | inactive | 2246 |
| 3 | 1294 | 18 |
| 4 | 6368 | 14 |
| 5 | inactive | 272 |
| 6 | 4580 | 355 |
| 7 | 720 | |
| 8 | 234 | 7 |
| 9 | 256 | 8 |
| 10 | 170 | 7 |
| 11 | 102 | |
| 12 | 71 | |
| 13 | 60 | 6 |
| 14 | | 85 |

Table 2 tested compounds: X varies and n is 13.

TABLE 2

X

| n = 13 | COOH | CH₂OH |
|---|---|---|
| R₁, R₂ = H | 60 | 149 |
| R₁ = NH₂, R₂ = H | 6.4 (DL) | |
| | 2.6 (L) | |
| R₁ = NH—CH₃, R₂ = H | 29.3 (DL) | |
| R₁ = NH₂, R₂ = CH₃ | 53.7 (DL) | |

Table 3 test compounds: R¹ is NH₂, R² is H and X varies.

TABLE 3

| | X | | |
|---|---|---|---|
| n | PO₃H₂ | S0₂H | SO₃H |
| 1 | 524 (DL) | 1152 (L) | 739 (L) |
| 2 | 505 (DL) | 224 (L) | 63 (L) |
| 3 | 1000 (DL) | | |
| 4 | | | |
| 5 | 1460 (DL) | | |

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of decreasing a level of kynurenic acid in a cell, comprising:
contacting a cell exhibiting kynurenine aminotransferase II activity with an amount of a dicarboxylic acid compound or a derivative or analog thereof effective to inhibit synthesis of kynurenic acid by said kynurenine aminotransferase II activity thereby decreasing the level of kynurenic acid in the cell, said dicarboxylic acid compound or said derivative or analog thereof having the structural formula: 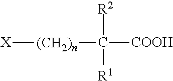
wherein R¹ is H, NH₂ or NHCH₃;
R² is H or CH₃;
n is 0 to 14; and
X is —COOH, —CH₂OH, —PO₃H₂, —SO₂H, or —SO₃H; or
a pharmacologically acceptable salt thereof.

2. The method of claim 1, wherein said dicarboxylic acid compound or derivative or analog thereof is 1,6-hexanedioic acid, 1,7-heptanedioic acid, 1,12-dodecanedioic acid, 1,13-tridecanedioic acid, 1,14-tetradecanedioic acid, 1,15-pentadecanedioic acid, 1,16-hexadecanedioic acid, 1,9-nonanedioic acid, 1,10-decanedioic acid, 1,11-undecanedioic acid, 16-hydroxyhexadecanoic acid, 2-amino-7-phosphonoheptanoic acid, cysteinesulfinic acid, homocysteinesulfinic acid, 2-amino-3-sulfopropionic acid, or 2-amino-4-sulfobutyric acid.

3. The method of claim 1, wherein said dicarboxylic acid derivative or analog is a racemate.

4. The method of claim 3, wherein said racemate is DL-2-amino-1,4-butanedioic acid, DL-2-amino-1,5-pentanedioic acid, DL-2-amino-1,6-hexanedioic acid, DL-2-amino-1,7-heptanedioic acid, DL-2-amino-1,8-octanedioic acid, DL-2-amino-1,9-nonanedioic acid, DL-2-amino-1,11-undecanedioic acid, DL-2-amino-1,12-dodecandioic acid, DL-2-amino-1,13-tridecanedioic acid, DL-2-amino-1,16-hexadecanedioic acid, DL-2-amino-2-methyl-1,16-hexadecanedioic acid, DL-2-methylamino-1,16-hexadecanedioic acid, DL-2-amino-1,17-heptadecanedioic acid, DL-2-amino-4-phosphonobutyric acid, DL-2-amino-5-phosphonopentanoic acid, or DL-2-amino-6-phosphonohexaanoic acid.

5. The method of claim 1, wherein said dicarboxylic acid derivative or analog is a levarotatory isomer.

6. The method of claim 5, wherein said levarotatory isomer is L-2-amino-1,16-hexadecanedioic acid.

7. The method of claim 1, wherein R² is H and X is COOH in said dicarboxylic acid compound or derivative thereof.

8. The method of claim 7, wherein said dicarboxylic acid compound is 1,6-hexanedioic acid, 1,7-heptanedioic acid, 1,12-dodecanedioic acid, 1,13-tridecanedioic acid, 1,14-tetradecanedioic acid, 1,15-pentadecanedioic acid, 1,16-hexadecanedioic acid, DL-2-amino-1,4-butanedioic acid, DL-2-amino-1,5-pentanedioic acid, DL-2-amino-1,6-hexanedioic acid, DL-2-amino-1,7-heptanedioic acid, DL-2-amino-1,8-octanedioic acid, DL-2-amino-1,9-nonanedioic acid, DL-2-amino-1,11-undecanedioic acid, 1,9-nonanedioic acid, 1,10-decanedioic acid, 1,11-undecanedioic acid, DL-2-amino-1,12-dodecandioic acid, DL-2-amino-1,13-tridecanedioic acid, DL-2-amino-1,16-hexadecanedioic acid, L-2-amino-1,16-hexadecanedioic acid, DL-2-amino-1,17-heptadecanedioic acid, or DL-2-methylamino-1,16-hexadecanedioic acid.

9. The method of claim 1, wherein R² is CH₃ and X is COOH in said dicarboxylic acid derivative.

10. The method of claim 9, wherein said dicarboxylic acid compound is DL-2-amino-2-methyl-1,16-hexadecanedioic acid.

11. The method of claim 1, wherein R¹ is H or NH₂ and R² is H in said dicarboxylic acid analog.

12. The method of claim 11, wherein said dicarboxylic acid analog is 16-hydroxyhexadecanoic acid, DL-2-amino-4-phosphonobutyric acid, DL-2-amino-5-phosphonopentanoic acid, DL-2-amino-6-phosphonohexaanoic acid, 2-amino-7-phosphonoheptanoic acid, cysteinesulfinic acid, homocysteinesulfinic acid, 2-amino-3-sulfopropionic acid, or 2-amino-4-sulfobutyric acid.

13. The method of claim 1, wherein the level of kynurenic acid is associated with a cognitive disease or disorder in a subject.

14. The method of claim 13, wherein said cognitive disease or disorder is mental retardation, is associated with Alzheimer's Disease, is associated with Parkinson's Disease, a neurodegenerative disease or seizure disorders, or an age-related cognitive deficit.

15. The method of claim 1, wherein the level of kynurenic acid is associated with a psychiatric disease or disorder in a subject.

16. The method of claim 15, wherein said psychiatric disease or disorder is schizophrenia, attention-deficit hyperactivity disorder, bipolar disease, depression, an obsessive-compulsive disorder, or drug addiction.

17. A method of treating a pathophysiological condition associated with an increase in kynurenic acid in a subject, comprising:
administering to the subject a pharmacologically effective amount of a compound having the structural formula: 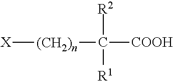
wherein R¹ is H, NH₂ or NHCH₃;
R² is H or CH₃;
n is 8 to 14; and
X is —COOH, —CH₂OH, —PO₃H₂, —SO₂H, or —SO₃H; or
a pharmacologically acceptable salt thereof.

18. The method of claim 17, wherein said dicarboxylic acid compound or derivative or analog thereof is 1,6-hexanedioic acid, 1,7-heptanedioic acid, 1,12-dodecanedioic acid, 1,13-tridecanedioic acid, 1,14-tetradecanedioic acid, 1,15-pentadecanedioic acid, 1,16-hexadecanedioic acid, 1,9-nonanedioic acid, 1,10-decanedioic acid, 1,11-undecanedioic acid, 16-hydroxyhexadecanoic acid, 2-amino-7-phosphonoheptanoic acid, cysteinesulfinic acid, homocysteinesulfinic acid, 2-amino-3-sulfopropionic acid, or 2-amino-4-sulfobutyric acid.

19. The method of claim 17, wherein said dicarboxylic acid derivative or analog is a racemate.

20. The method of claim 19, wherein said racemate is DL-2-amino-1,4-butanedioic acid, DL-2-amino-1,5-pentanedioic acid, DL-2-amino-1,6-hexanedioic acid, DL-2-amino-1,7-heptanedioic acid, DL-2-amino-1,8-octanedioic acid, DL-2-amino-1,9-nonanedioic acid, DL-2-amino-1,11-undecanedioic acid, DL-2-amino-1,12-dodecandioic acid, DL-2-amino-1,13-tridecanedioic acid, DL-2-amino-1,16-hexadecanedioic acid, DL-2-amino-2-methyl-1,16-hexadecanedioic acid, DL-2-methylamino-1,16-hexadecanedioic acid, DL-2-amino-1,17-heptadecanedioic acid, DL-2-amino-4-phosphonobutyric acid, DL-2-amino-5-phosphonopentanoic acid, or DL-2-amino-6-phosphonohexaanoic acid.

21. The method of claim 17, wherein said dicarboxylic acid derivative or analog is a levarotatory isomer.

22. The method of claim 21, wherein said levarotatory isomer is L-2-amino-1,16-hexadecanedioic acid.

23. The method of claim 22, wherein said L-2-amino-1,16-hexadecanedioic acid comprises a pharmaceutical composition including a pharmaceutically acceptable carrier.

24. The method of claim 17, wherein R² is H and X is COOH in said dicarboxylic acid compound or derivative thereof.

25. The method of claim 24, wherein said dicarboxylic acid compound is 1,6-hexanedioic acid, 1,7-heptanedioic acid, 1,9-nonanedioic acid, 1,10-decanedioic acid, 1,11-undecanedioic acid, 1,12-dodecanedioic acid, 1,13-tridecanedioic acid, 1,14-tetradecanedioic acid, 1,15-pentadecanedioic acid, or 1,16-hexadecanedioic acid, DL-2-amino-1,4-butanedioic acid, DL-2-amino-1,5-pentanedioic acid, DL-2-amino-1,6-hexanedioic acid, DL-2-amino-1,7-heptanedioic acid, DL-2-amino-1,8-octanedioic acid, DL-2-amino-1,9-nonanedioic acid, DL-2-amino-1,11-undecanedioic acid, DL-2-amino-1,12-dodecandioic acid, DL-2-amino-1,13-tridecanedioic acid, DL-2-amino-1,16-hexadecanedioic acid, L-2-amino-1,16-hexadecanedioic acid, DL-2-methyamino-1,16-hexadecanedioic acid, or DL-2-amino-1,17-heptadecanedioic acid.

26. The method of claim 17, wherein R² is CH₃ and X is COOH in said dicarboxylic acid derivative.

27. The method of claim 26, wherein said dicarboxylic acid compound is DL-2-amino-2-methyl-1,16-hexadecanedioic acid.

28. The method of claim 17, wherein R¹ is H or NH₂ and R² is H in said dicarboxylic acid analog.

29. The method of claim 28, wherein said dicarboxylic acid analog is 16-hydroxyhexadecanoic acid, DL-2-amino-4-phosphonobutyric acid, DL-2-amino-5-phosphonopentanoic acid, DL-2-amino-6-phosphonohexaanoic acid, 2-amino-7-phosphonoheptanoic acid, cysteinesulfinic acid, homocysteinesulfinic acid, 2-amino-3-sulfopropionic acid, or 2-amino-4-sulfobutyric acid.

30. The method of claim 17, wherein said administered compound comprises a pharmaceutical composition including a pharmaceutically acceptable carrier.

31. The method of claim 17, wherein the pathophysiological condition is a cognitive disease or disorder.

32. The method of claim 31, wherein said cognitive disease or disorder is mental retardation, is associated with Alzheimer's Disease, is associated with Parkinson's Disease, a neurodegenerative disease or seizure disorders, or an age-related cognitive deficit.

33. The method of claim 17, wherein the pathophysiological condition is a psychiatric disease or disorder.

34. The method of claim 33, wherein said psychiatric disease or disorder is schizophrenia, attention-deficit hyperactivity disorder, bipolar disease, depression, an obsessive compulsive disorder or drug addiction.

35. A method for identifying an inhibitory compound of kynurenine aminotransferase II activity, comprising:
selecting a test compound;
measuring the level of kynurenic acid in the presence or absence of the test compound; and
comparing the level of kynurenic acid in the presence of the test compound with the level of kynurenic acid in the absence of the test compound, wherein a decrease in kynurenic acid in the presence of the test compound is indicative that the test compound inhibits kynurenine aminotransferase II activity.

36. The method of claim 35, wherein said test compound has the structural formula: 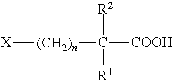
wherein R¹ is H, NH₂ or NHCH₃;
R² is H or CH₃;
n is 0 to 14; and
X is —COON, —CH₂OH, —PO₃H₂, —SO₂H, or —SO₃H; either as the racemate or levorotatory isomer thereof.

37. The method of claim 35, further comprising:
reducing levels of kynurenic acid associated with a cognitive disease or disorder in a subject.

38. The method of claim 37, wherein said cognitive disease or disorder is mental retardation, is associated with Alzheimer's Disease, is associated with Parkinson's Disease, a neurodegenerative disease or seizure disorders, or an age-related cognitive deficit.

39. The method of claim 35, further comprising:
reducing levels of kynurenic acid associated with a psychiatric disease or disorder in a subject.

40. The method of claim 39, wherein said psychiatric disease or disorder is schizophrenia, attention-deficit hyperactivity disorder, bipolar disease, depression, an obsessive compulsive disorder or drug addiction.

41. An inhibitory compound identified by the method of claim 35.

42. A pharmaceutical composition comprising the inhibitory compound of claim 41 and a pharmaceutically acceptable carrier.
